(19) United States
(12) Patent Application Publication
(10) Pub. No.: US 2015/0359387 A1
(43) Pub. Date: Dec. 17, 2015

(54) UTENSIL RETRACTOR

(71) Applicant: ServePur Inc., Encinitas, CA (US)

(72) Inventor: Andre Ramirez, Encinitas, CA (US)

(73) Assignee: ServePur Inc., Encinitas, CA (US)

(21) Appl. No.: 14/717,968

(22) Filed: May 20, 2015

Related U.S. Application Data
(60) Provisional application No. 62/011,477, filed on Jun. 12, 2014.

(51) Int. Cl. A47J 43/28; F16B 1/00; G01N 33/02; F16M 13/02; G01K 13/00; F16B 2/02; F16B 47/00

(52) U.S. Cl. CPC A47J 43/287; F16B 2/02; F16B 1/00; F16B 47/00; F16M 13/02; F16M 13/022; G01K 13/00; G01N 33/02; F16B 2001/0035

(57) ABSTRACT

A utensil retracting device is described that can assist with serving food and storing a utensil used for serving the food. For example, the utensil retracting device can be secured to a part of a food service area and can also be secured to a utensil that can be used for serving food. The utensil retracting device can secure the utensil in a desired storing position and/or area. In addition, the utensil can be used within a range defined by a length of a tether that secures the utensil to the utensil retracting device. The utensil retracting device can also retract the tether in order to return and secure the utensil back to the desired storing position and/or area. The utensil retracting device can assist in preventing food contamination, such as from direct contact with diners as a result of misplaced utensils.

### CROSS-REFERENCE TO RELATED APPLICATION

The current application claims priority under 35 U.S.C. §119(e) to U.S. Provisional patent application Ser. No. 62/011477, filed on Jun. 12, 2014 and entitled “Retractable Line Mechanism & Cradle for Food Serving Utensils,” which is incorporated by reference herein in its entirety.

### TECHNICAL FIELD

The subject matter described herein relates to a utensil retracting device that can be secured to a variety of locations, such as a part of a food service area, for securing a utensil in a desired storage position or area and allowing the utensil to be retractably distanced from a part of the utensil retracting device.

### BACKGROUND

Establishments that serve food can include buffet or self-serve stations where diners can select various types of food for eating. In order to prevent the spread of germs and to assist the diners in selecting food, various utensils, such as tongs and forks, are provided for diners to use when placing the selected food onto a serving item (e.g. a plate, a bowl, etc.). However, such utensils can themselves become a source for spreading germs when they fall into the food, particularly when the hand-gripped portion of the utensil touches the food. This can be a result of the utensil falling or being placed directly back into or onto the food. When an unattached or unsecured utensil falls into a food pan, for example, a diner may insert a potentially unsanitary hand into the food pan, and thereby contact food contained in the food pan, to retrieve the utensil. In addition, such a utensil can be lost or misplaced, thereby causing a diner to select food from the buffet or self-serve station using either his or her (potentially unsanitary) hand or a utensil intended for use with other food. Use of a utensil to serve food other than that for which it is intended can result in various forms of contamination to the food in addition to customer inconvenience and dissatisfaction. Some health department regulations can require one specific serving utensil per food pan to prevent cross contamination and allergic reactions. An unattached or unsecured serving utensil, for example, can travel from a serving pan containing tuna to one containing sprouts or from one containing chicken to one containing lettuce, which can result in cross contamination, a diner having an allergic reaction, or other unfavorable outcomes.

Existing approaches relating to prevention of misplacement or contamination of serving utensils generally have one or more undesirable aspects. For example, a system for securing serving utensils that relies on tethering the utensils to an overhead point (e.g. example the sneeze guard of a buffet line, etc.) can actually lead to increased potential for serving utensil contamination or misuse. As an example, suspension of a serving utensil in this manner can make the handle portion of the serving utensil less convenient for a diner to grab, which can lead to a part of the utensil meant to contact food being touched by the diner in the process of trying to pick up the serving utensil.

### SUMMARY

Aspects of the current subject matter can relate to a utensil retracting device for assisting with serving food, such as at a buffet or self-serve food stations, and for storing a utensil used for serving the food.

In one aspect, a utensil retracting device includes a base configured for securing to a part of a food service area and allowing a utensil to be retractably distanced from the base. The utensil retracting device can include a retractor coupled to the base and include an elongated tether. The retractor can be configured to retract the elongated tether after allowing the elongated tether to be extended from the retractor. The elongated tether can include a proximal end that is secured to the retractor. A utensil adaptor can be secured at a distal end of the elongated tether. The utensil adaptor can be configured to releasably secure the utensil to the elongated tether.

In some variations one or more of the following features can optionally be included in any feasible combination. The base can include a securing feature, and the securing feature can include at least one of a clasp, a clamp, a magnet, a suction cup, and a threaded attachment. The securing feature can include a magnet and a magnet coupling feature configured to be positioned opposite the magnet such that the magnet attracts the magnet coupling feature through the part of the food service area. The food service area can include one or more of a metal food container, a wall, and a support structure. The elongated tether can have a length when extended that is sufficient to allow a user to effectively use the utensil coupled to the elongated tether, such as to select food from the food service area and to put the selected food onto a serving item. The utensil retracting device can also include a sensor that is configured to generate data relating to food contained within the food service area and a wireless communication device that is configured to transmit the data to a computing device. The sensor can include a temperature sensor and the data can include temperature data.

In another interrelated aspect of the current subject matter, a method includes securing a base of a utensil retracting device to a part of a food service area. The base can include a retractor configured to retract an elongated tether after allowing the elongated tether to extend from the retractor. The elongated tether can include a proximal end that is secured to the retractor and a distal end that is secured to a utensil. The method can further include allowing the elongated tether to extend from the retractor as the utensil is positioned a distance from the base, with the distance being confined within a length of the elongated tether. Additionally, the method can include retracting the elongated tether and thereby positioning the utensil in a storing orientation or area.

In yet another interrelated aspect, a method can include generating data relating to food contained within the food service area using a sensor coupled to the base and transmitting the generated data to a computing system.

In some variations one or more of the following features can optionally be included in any feasible combination. The securing can include magnetically coupling with a magnet and a magnet coupling feature positioned opposite the magnet such that the magnet attracts the magnet coupling feature through the part of the food service area. The securing can include use of a securing feature including at least one of a clasp, a clamp, a suction cup, and a threaded attachment. The method can further include sensing, with a sensor coupled to the base, data related to food contained within the food service area and transmitting the sensed data to a computing device. The data can include temperature data, the sensor can include a temperature sensor, and the base can include the sensor. The transmitting can include wirelessly transmitting using a wireless communication device. The base can include a machine-readable storage device and the data can be stored using the machine-readable storage device. The method can further include allowing the elongated tether to extend from the retractor as the utensil is positioned a distance from the base, with the distance being configured within a length of the elongated tether. The method can also include retracting the elongated tether and thereby positioning the utensil in a storing orientation or area.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 shows a diagram illustrating features of a utensil retracting device consistent with implementations of the current subject matter;

FIG. 2 shows a cross section of the utensil retracting device shown in FIG. 1. showing a retractor and a securing feature of the utensil retracting device;

FIG. 3A shows a front perspective view of the utensil retracting device of FIG. 1 coupled to a food serving container;

FIG. 3B shows a back perspective view of the utensil retracting device of FIG. 1 coupled to a food serving container;

FIG. 4 shows a process flow chart illustrating features of a method consistent with implementations of the current subject matter; and

FIG. 5 shows a process flow chart illustrating features of another method consistent with implementations of the current subject matter.

When practical, similar reference numbers denote similar structures, features, or elements.

### DETAILED DESCRIPTION

Implementations of the current subject matter can include a utensil retracting device for assisting with serving food and storing a utensil used for serving the food as well as methods of making and using such a device. For example, the utensil retracting device can be secured to a part of a food service area (e.g., a food pan) and can also be secured to a food serving utensil (e.g., serving spoon, tongs, etc.). When the utensil is not in use, the utensil retracting device can secure the utensil in a desired storing position and/or area. In addition, when the utensil is being used, such as for selecting food and placing the selected food onto a serving item (e.g. a plate, a bowl, etc.), the utensil retracting device can allow the utensil to be used within a range defined by a length of a tether that secures the utensil to a part of the utensil retracting device. When a user of the utensil (e.g. a diner) finishes using the utensil, the utensil retracting device can retract the tether in order to return and secure the utensil back to a storing position and/or area.

The storing position can include positioning the utensil in an orientation such that it is easy and intuitive for a user to grasp an appropriate part of the utensil, such as a part of the utensil that does not come into contact with food (e.g. a handle, a grip, etc.). In addition, the desired storing area can include an area that is sanitary for keeping the utensil clean and/or in an area or position that allows the user to grasp the utensil without contaminating the food. As such, securing the utensil in the desired storing position and/or area can promote sanitary conditions, such as by preventing the utensil from falling into the food, and allow users to easily access the utensil for use.

In some implementations, the utensil retracting device can include a base that is configured to secure to one or more of a variety of locations associated with a food service area. The base can include a retractor configured to allow an elongated tether to extend from the retractor as well as to retract the tether back to the retractor. In addition, a utensil adapter can secure a utensil to a distal end of the tether. As such, the utensil can be retractably distanced from the base of the utensil retracting device by a distance defined by a length of the tether. The retractor can retract the tether along with the utensil that is attached to the tether, such as after use of the utensil in order to place the utensil in a desired storing position or area.

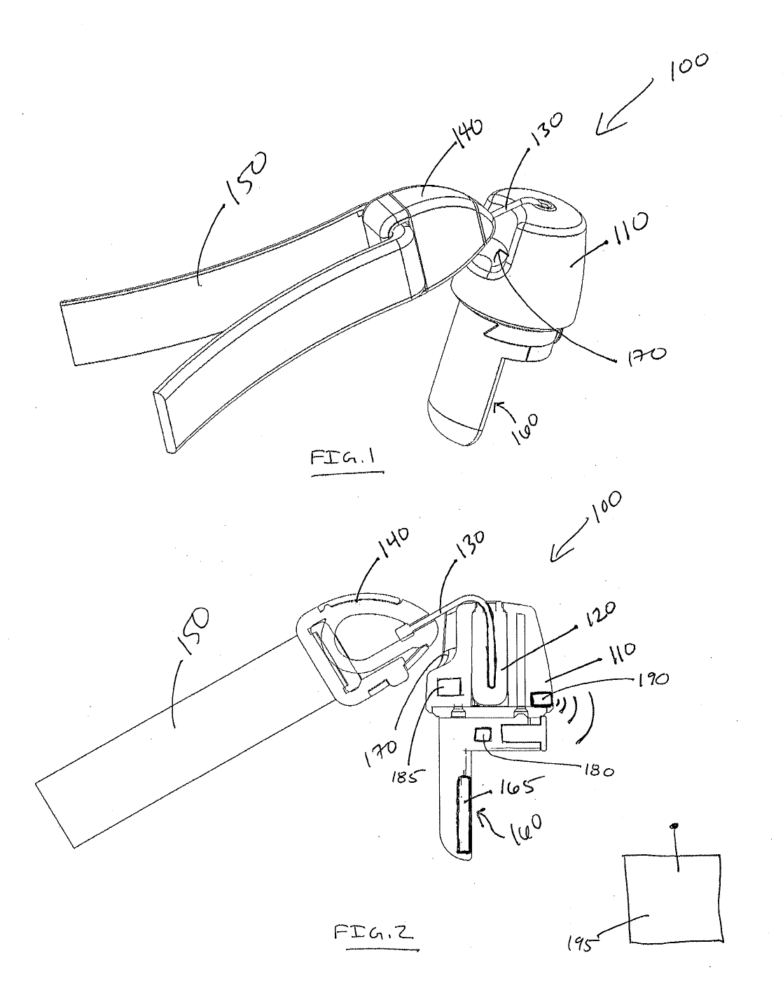

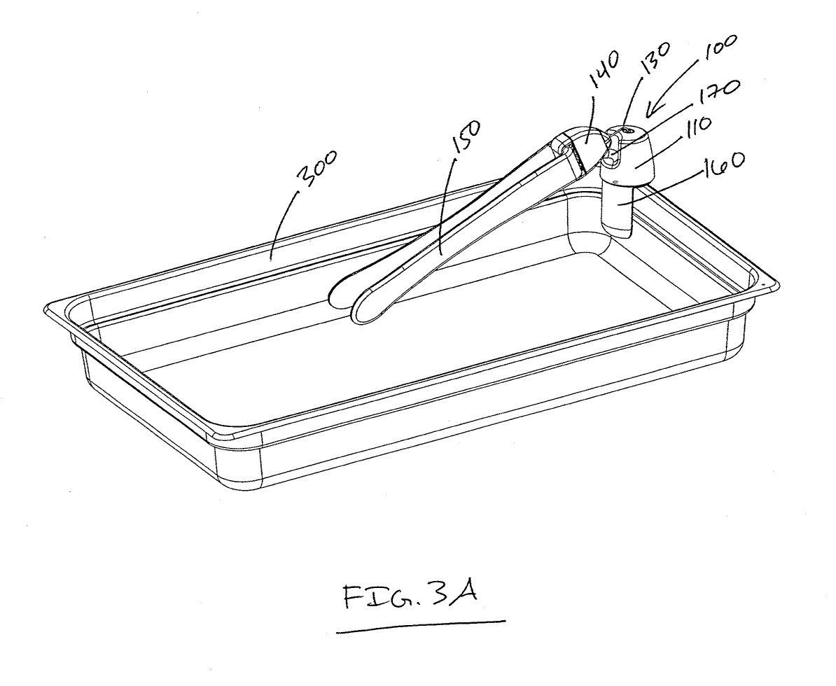

FIGS. 1 and 2 show two views illustrating features of an implementation of a utensil retracting device 100 that includes a base 110, a retractor 120 including an elongated tether 130, and a utensil adapter 140. The utensil adapter 140 can be coupled to a utensil 150 and to a distal end of the tether 130. The retractor 120 can allow the utensil 150 to be extended from the retractor 120 or the base 110 a distance defined by a length of the tether 130. The retractor 120 can also retract the tether 130, thereby placing the utensil 150 in a desired storing position or area.

The base 110 can include a securing feature 160 that can assist with securing a part of the utensil retracting device 100 to a part of a food service area. As shown in FIG. 2, the securing feature 160 can include a magnet 165, which can assist with magnetically securing the base 110 of the utensil retracting device 100 to a part of the food service area.

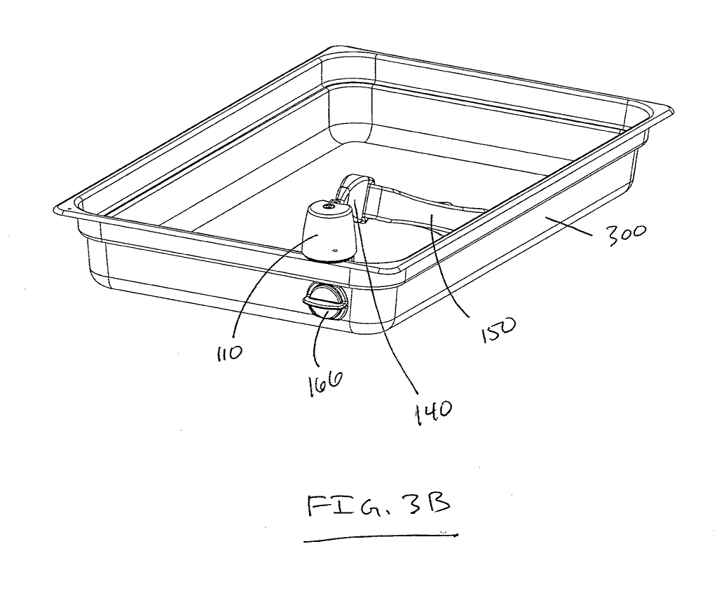

FIGS. 3A and 3B illustrate an example of the base 110 of the utensil retracting device 100 magnetically secured to a metal food container 300. The magnet 165 can be positioned within the base 110 and can provide sufficient magnetic attraction for allowing the base 110 to be releasably secured to the metal food container 300. As shown in FIG. 3B, the utensil retracting device 100 can further include a magnet coupling feature 166 that is configured to magnetically couple to the magnet 165 in order to secure the base 110 to a part of a food service area, including to a container 300, which need not be metal or otherwise attractive to the magnet 165. Rather, the magnet 165 and the magnet coupling feature 166 can be disposed on opposite sides of the part of the food service area such that the magnet 165 attracts the magnet coupling feature 166 through the part of the food service area (e.g. a non-metallic food tray, etc.). For example, the magnet coupling feature 166 can magnetically couple to the magnet 165 even with the part of the food service area positioned between the magnet coupling feature 166 and the magnet 165. In some implementations, a user can remove the magnet coupling feature 166 from the part of the food service area in order to disengage the base 110 from the part of the food service area.

The base 110 can be configured in various shapes and sizes that allow the base 110 to adapt to a variety of locations within and adjacent to the food service area. For example, although FIGS. 3A and 3B show the utensil retracting device 100 coupled to a metal food container 300, the base 110 of the utensil retracting device 100 can be coupled to a variety of locations, including a wall or support structure adjacent the food service area. Alternatively or in addition, the securing feature 160 can include one or more of a variety of features for assisting with securing the base 110 of the utensil retracting device 100 to a part of the food service area, such as one or more of a clasp, a clamp, a magnet, a suction cup, a threaded attachment, etc. In one example, a magnetic securing feature can be used with a non-metallic part of the food service area by inclusion of a metallic part configured to be positioned opposite the magnetic part of the securing feature with the non-metallic part of the food service area disposed between the magnetic part and the metallic part.

In some implementations, the base 110 can include a cradle 170 (as shown in FIGS. 1-2) including a cutout along a side of the base 110, which can accept either a part of the utensil 150 or part of the utensil adapter 140, such as when the utensil 150 is not in use. For example, when the tether 130 is in the retracted position, the utensil adapter 140 can be positioned within the cradle 170, which can position the utensil 150 for easy access and use by a user. The cradle 170 can be positioned in a variety of locations along the base 110, and the cradle 170 can include a variety of shapes and sizes, such as for accommodating a variety of utensils 150 in a variety of positions.

The retractor 120 can be secured to the base 110 in a variety of ways, including contained within the base 110, as shown in FIG. 2. The retractor 120 can be secured to a proximal end of the tether 130 and allow the tether 130 to extend from the retractor 120 no more than a distance defined by the length of the tether. In addition, the retractor 120 can also be configured to retract the tether 130, along with anything coupled to the tether 130, back towards the retractor 120. For example, the retractor 120 can retract the tether 130 with a sufficient pulling force to be able to move the utensil 150 coupled to the tether 130 to the storing position or area.

The tether 130 can be made out of one or more of a variety of materials (e.g. plastic, metal wire, natural or synthetic threads, etc.), and can have one or more of a variety of shapes and sizes. For example, the tether 130 can have a length when extended that is sufficient to allow a user to effectively use the utensil 150 coupled to the tether 130, such as for selecting food and putting the selected food onto a serving item. In some implementations, the tether 130 can have a length such that in an extended position (i.e., fully extended from the retractor), the utensil 150 coupled to the tether 130 can be positioned approximately 0.5 foot to approximately 3 feet away from the base 110 of the utensil retracting device 100.

The utensil adaptor 140 can releasably secure a utensil 150, which can allow interchanging of different utensils 150 coupled to the utensil retracting device 100, and can also allow easy cleaning of the utensils 150. For example, the utensil adapter 140 can include a clamp that can attach to a one or more of a variety of utensils 150. Some implementations of the utensil adapter 140 can include a push button (or control or similar feature) to allow for a quick-release of a utensil 150 that is coupled or otherwise attached to the utensil adapter 140. Although examples of the utensil adapter 140 are provided herein that include a clamp and/or a push button, the utensil adapter 140 can include any of a variety of features that allow the utensil adapter 140 to releasably attach to a variety of utensils 150. The utensil adapter 140 can also include a gripping portion that is made out of a material that can assist with grasping and manipulating the utensil adapter 140, such as a rubber material or the like.

In some implementations of the current subject matter, one or more parts of the utensil retracting device 100 can be made out of an anti-microbial material or any material that is beneficial in food service environments, such as plastic, stainless steel, and the like. Furthermore, the utensil retracting device 100 can be machine washable such that the entire utensil retracting device 100 can be placed in a dish washer for cleaning and sterilizing.

As shown in FIG. 2, some implementation of the utensil retracting device 100 can include one or more sensors 180, such as for example a temperature sensor for collecting temperature measurements from food provided in the food service area. In addition, the utensil retracting device 100 can include a data storage device 185 that is configured to store the data collected by the one or more sensors. Additionally, the utensil retracting device 100 can include a wireless communication device 190 (e.g., Wi-Fi, Bluetooth, zigbee, etc.) for allowing data to be sent to a remote server 195, such as data collected by the one or more sensors 180 or stored on the data storage device 185. In some implementations of the current subject matter, a wireless communication device 190 can be included, and this wireless communication device 190 can be configured to periodically (or optionally continuously, semi-continuously, etc.) transmit data from the one or more sensors 180 to an external system or systems (e.g., remote server 195) that includes computer hardware and/or one or more data storage devices. Incorporation of such features can allow, for example, temperature readings of the food provided in the food service area to be recorded, tabulated, analyzed, reported, etc., such as for ensuring compliance with food service regulations that require food to be kept within a defined temperature range. Any one of the one or more sensors 180, data storage device 185, and wireless communication device 190 can be located in or coupled to the base 110 of the utensil retracting device 100. However, the one or more sensors 180, data storage device 185, and/or wireless communication device 190 can be located in a variety of locations within and/or adjacent the utensil retracting device 100.

In some implementations, prior to the utensil retracting device 100 being used the tether 130 can be in the retracted position, which can securely place the utensil 150 in the desired storage position or area. When a user grasps and pulls the utensil 150, such as for selecting a food item, the retractor 120 can allow the tether 130 to extend from the retractor 120. The retractor 120 can maintain a tension on the tether 130 such that the user can easily manipulate the utensil 150 while also preventing slack in the tether 130. In addition, the tension in the tether 130 can also allow the retractor 120 to immediately retract the tether 130, including the utensil 150 coupled to the tether 130, upon the user's release of the utensil 150. Additionally, the retractor 120 can continue to retract the tether 130 until the tether 130 has returned to the retracted position and either the utensil 150 or utensil adapter 140 is positioned in a desired position or area (e.g., within the cradle).

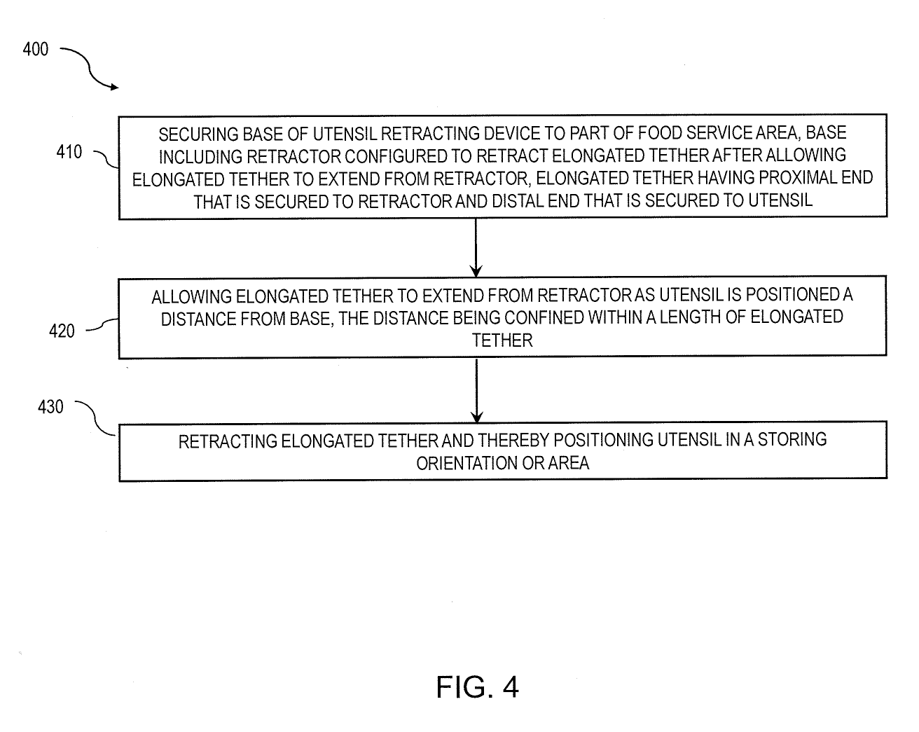

FIG. 4 illustrates a data flow diagram showing features that can be included in a method associated with the utensil retracting device described herein. For example, at 410, a base of a utensil retracting device can be secured to a part of a food service area. The base can include a retractor configured to retract an elongated tether after allowing the elongated tether to extend from the retractor. In addition, the elongated tether can include a proximal end that is secured to the retractor and a distal end that is secured to a utensil. At 420, the elongated tether can be allowed to extend from the retractor as the utensil is positioned a distance from the base, which can be confined within a length of the elongated tether. At 430, the elongated tether can be retracted thereby positioning the utensil in a storing orientation or area.

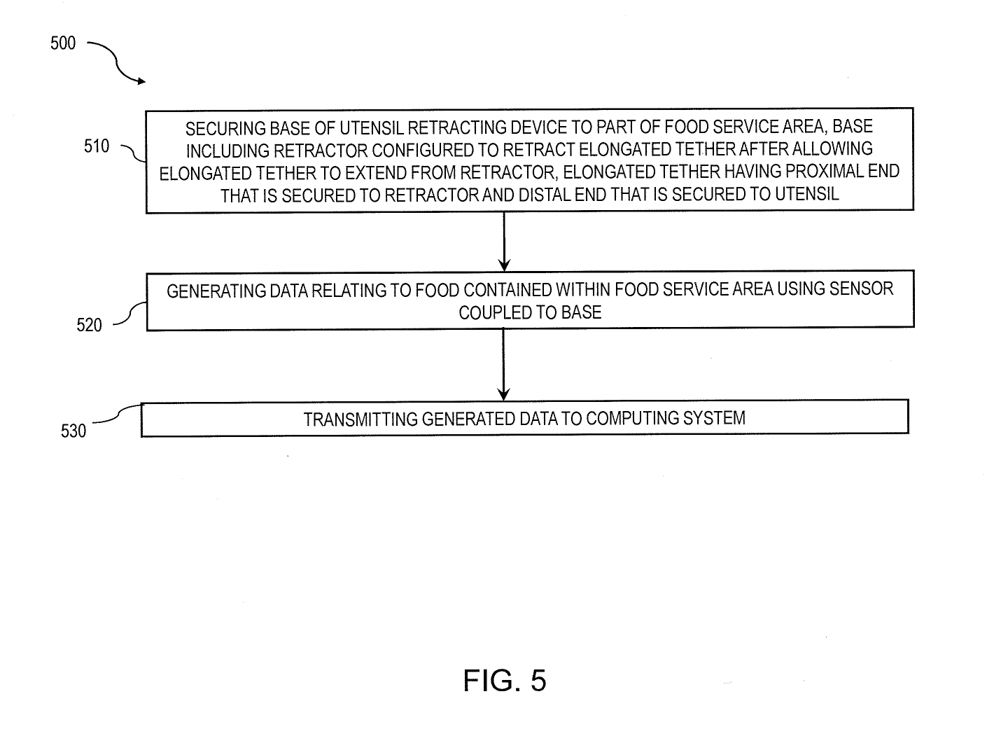

FIG. 5 illustrates a process flow diagram showing features that can be included in another method associated with the utensil retracting device described herein. For example, at 510, a base of a utensil retracting device can be secured to a part of a food service area. The base can include a retractor configured to retract an elongated tether after allowing the elongated tether to extend from the retractor. In addition, the elongated tether can include a proximal end that is secured to the retractor and a distal end that is secured to a utensil. At 520, data relating to food contained within the food service area using a sensor coupled to the base can be generated. At 530, the generated data can be transmitted to a computing system.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A utensil retracting device comprising;
a base configured to be secured to a part of a food service area;
a retractor coupled to the base and including an elongated tether, the retractor configured to retract the elongated tether after allowing the elongated tether to be extended from the retractor, the elongated tether having a proximal end that is secured to the retractor; and
a utensil adaptor secured at a distal end of the elongated tether, the utensil adaptor configured to releasably secure a serving utensil to the elongated tether.

2. The utensil retractor of claim 1, wherein the base comprises a securing feature.

3. The utensil retracting device of claim 2, wherein the securing feature comprises at least one of a clasp, a clamp, a magnet, a suction cup, and a threaded attachment.

4. The utensil retracting device of claim 2, wherein the securing feature comprises a magnet and a magnet coupling feature configured to be positioned opposite the magnet such that the magnet attracts the magnet coupling feature through the part of the food service area.

5. The utensil retracting device of claim 1, wherein the part of the food service area comprises one or more of a metal food container, a wall, and a support structure.

6. The utensil retracting device of claim 1, wherein the elongated tether has a length when extended that is sufficient to allow a user to effectively use the utensil coupled to the elongated tether to select food from the food service area and to put the selected food onto a serving item.

7. The utensil retracting device of claim 1, further comprising:
a sensor configured to generate data relating to food contained within the food service area; and
a wireless communication device configured to transmit the data to a computing device.

8. The utensil retracting device of claim 7, wherein the sensor comprises a temperature sensor and the data comprises temperature data.

9. A method comprising;
securing a base of a utensil retracting device to a part of a food service area, the base including a retractor configured to retract an elongated tether after allowing the elongated tether to extend from the retractor, the elongated tether having a proximal end that is secured to the retractor and a distal end that is secured to a utensil;
allowing the elongated tether to extend from the retractor as the utensil is positioned a distance from the base, the distance being confined within a length of the elongated tether; and
retracting the elongated tether and thereby positioning the utensil in a storing orientation or area.

10. The method of claim 9, wherein the securing comprises magnetically coupling with a magnet and a magnet coupling feature positioned opposite the magnet such that the magnet attracts the magnet coupling feature through the part of the food service area.

11. The method of claim 9, wherein the securing comprises use of a securing feature including at least one of a magnet, a clasp, a clamp, a suction cup, and a threaded attachment.

12. The method of claim 9, further comprising:
sensing, with a sensor coupled to the base, data related to food contained within the food service area; and
transmitting the sensed data to a computing device.

13. The method of claim 12, wherein the sensor comprises a temperature sensor and the data comprises temperature data.

14. A method comprising;
securing a base of a utensil retracting device to a part of a food service area, the base including a retractor configured to retract an elongated tether after allowing the elongated tether to extend from the retractor, the elongated tether having a proximal end that is secured to the retractor and a distal end that is secured to a utensil;
generating data relating to food contained within the food service area using a sensor coupled to the base; and
transmitting the generated data to a computing system.

15. The method of claim 14, wherein the data comprise temperature data, and wherein the sensor comprises a temperature sensor.

16. The method of claim 14, wherein the base comprises the sensor.

17. The method of claim 14, wherein the base comprises a machine-readable storage device, and wherein the method further comprises storing the data using the machine-readable storage device.

18. The method of claim 14, wherein the base comprises a securing feature configured to assist with the securing the base to the part of the food service area, the securing feature including at least one of a clasp, a magnet, clamp, a suction cup, and a threaded attachment.

19. The method of claim 14, further comprising:
allowing the elongated tether to extend from the retractor as the utensil is positioned a distance from the base, the distance being confined within a length of the elongated tether; and
retracting the elongated tether and thereby positioning the utensil in a storing orientation or area.

20. The method of claim 14, wherein the transmitting comprises wirelessly transmitting using a wireless communication device.
